(19) United States
(12) Patent Application Publication
(10) Pub. No.: US 2009/0312827 A1
(43) Pub. Date: Dec. 17, 2009

(54) BALLOON CATHETER WITH CENTRALIZED VENT HOLE

(75) Inventor: Corey E. Stapleton, Gilbert, AZ (US)

Correspondence Address: C. R. Bard, Inc.;Bard Peripheral Vascular, Inc., 1415 W. 3rd Street, P.O. Box 1740, Tempe, AZ, 85280-1740, US

(73) Assignee: C.R. Bard Inc., Murray Hill, NJ (US)

(21) Appl. No.: 12/096,773

(22) PCT Filed: Dec. 18, 2006
(86) PCT No.: PCT/US2006/062232

§ 371 Date: Aug. 27, 2008

Related U.S. Application Data
(60) Provisional application No. 60/752,878, filed on Dec. 23, 2005.

(51) Int. Cl. A61F 2/84; A61M 25/10

(52) U.S. Cl. 623/1.11; 604/103.1; 604/500

(57) ABSTRACT

A system and method providing a catheter assembly for engaging a stenosis. The assembly includes a catheter defining a first lumen and a second lumen spaced apart and disposed about a longitudinal axis. The catheter includes an opening in communication with the first lumen to define a flow path having an angle incident to the longitudinal axis. A first marker; and a second marker disposed on the catheter are spaced equidistantly from the opening. The assembly includes a balloon having a first end and a second end each sealed about the catheter and equidistantly from the opening to define a holding volume therebetween. The opening is disposed within the holding volume thereby placing the first lumen in sealed fluid communication with the holding volume. In a preferred embodiment, the catheter assembly includes a stent disposed about the balloon, and the balloon is configured to engage the stent with a stenosis.

### PRIORITY DATA AND INCORPORATION BY REFERENCE

This application claims benefit of priority to U.S. Provisional Patent Application No. 60/752,878 filed Dec. 23, 2005 which is incorporated by reference in its entirety.

### TECHNICAL FIELD

The present invention relates generally to balloon catheter assemblies for use in angioplasty and stent delivery procedures. In particular, the present invention provides a system and method for delivery of a balloon catheter to a stenosed blood vessel and inflation of the dilation balloon to expand a stent implant and/or the stenosed blood vessel.

### BACKGROUND ART

A large number of balloon catheters have been devised for angioplasty and stent delivery procedures. Commonly a guide wire is first introduced percutaneously into the patient's vascular system, advanced and then steered to the site of a stenosis. A dilation balloon or catheter is then advanced over the guide wire until the balloon is positioned within the stenosis so that on inflation, the balloon will compress the stenosis by dilatation of the blood vessel to thereby re-establish a more adequate blood flow path past the stenosis. To facilitate even compression pressure distribution along the length of the stenosed lesion, it is preferred that the dilation balloon be centered relative to the stenosis so as to fully engage the lesion.

Balloon dilation catheters have also been utilized in stent delivery in which the stent is disposed about the balloon and inflated into place at the stenosis. Catheter operators seek accurate deployment of the stent directly on the diseased tissue of the vessel in order to avoid stent migration to either side of the diseased tissue thereby avoiding or minimizing the chance of leaving some of the diseased tissue untreated. Accurate stent deployment is also desirable in order to avoid adversely affecting healthy tissue.

Stent misplacements may occur because of specific inflation dynamics experienced by the expandable balloon when deploying the stent. Known stent delivery catheters inflate the balloon portion of the catheter preferentially from either the distal or proximal end of the balloon. During inflation, the expanding balloon may form an unsymmetrical growth or inflation wave that may be said to drive or plow the stent so that it opens progressively from one end to the other along the front of the inflation wave. The wave may sometimes cause the stent to disengage prematurely from the balloon. This form of balloon inflation is referred to as “end-to end” preferential inflation. End-to-end balloon inflation may further cause a deploying stent to displace longitudinally away from its intended delivery site, thereby potentially ineffectively treating the diseased lesion within the patient's vasculature.

Known balloon dilation catheters used in connection with stent deployment and/or other applications are shown and described in several U.S. Patents including: U.S. Pat. Nos. 6,136,011; 5,908,448; 5,226,880; 5,176,619; 4,811,737; 5,409,495; 5,334,148; 5,169,386; and 3,939,820. In U.S. Pat. No. 6,592,568, described is one inflation technique for medial inflation of the balloon using an intermediate balloon inside a stent delivering dilation balloon to concentrate a bolus of fluid medially for distribution through the dilation balloon. The intermediate balloon can either be rupturable or otherwise provide a controlled fluid leak to release fluid into the dilation balloon. This technique, however, adds complexity to the procedure by requiring controlled bursting or leakage of an intermediate balloon.

Another complex stent delivery and deployment device is shown and described in U.S. Pat. No. 6,203,558 in which a stent is disposed about an inflation balloon. The inflation balloon is disposed about a catheter assembly having an inner shaft and an outer shaft. The inflation balloon is inflated from its proximal end by the delivery of a pressurized fluid flowing between the inner and outer shafts. The deployment device also includes an expandable securement device disposed about the inner shaft and disposed within the inflation balloon. The inner shaft has a single lumen for carrying a guide wire and fluid for expanding the securement device. To expand the securement member, fluid is discharged from the single lumen through a valve disposed along the inner shaft and centrally located within the securement member. For example, see FIG. 34 of the '558 patent. The expanded securement member secures the engagement between the inflation balloon and the stent.

Another patent, U.S. Pat. No. 6,648,854, also discloses a single lumen balloon tipped catheter for inflating a balloon having an operating pressure of about one atmosphere. The catheter effectively utilizes a single lumen to carry both a guide wire and inflation fluid. However, where balloons having higher operating pressures are utilized, a single lumen device may not be sufficient to provide the adequate pressure for inflating the balloon.

### DISCLOSURE OF INVENTION

A preferred embodiment according to the present invention provides a catheter assembly for engaging a stenosis. The assembly includes a catheter including a wall having a proximal end and a distal end along a longitudinal axis. The wall preferably has an interior surface and an exterior surface, in which the interior surface defines a first lumen and a second lumen spaced apart and disposed about the longitudinal axis. The wall preferably defines an opening extending between the interior surface and the exterior surface. The opening is in communication with the first lumen to define a flow path having an angle incident to the longitudinal axis. The exterior surface further preferably includes a first radiopaque and/or radiographic marker; and a second radiopaque and/or radiographic marker spaced apart from one another along the longitudinal axis so as to be substantially equidistant from the opening. The assembly also preferably includes a balloon having a first end and a second end defining a holding volume therebetween. The first end and the second ends are preferably sealed about the exterior surface. The opening is disposed within the holding volume thereby placing the first lumen in sealed fluid communication with the holding volume. The first and second ends of the balloon are further preferably spaced substantially equidistantly about the opening along the longitudinal axis.

Applicant recognizes that it is desirable to have an apparatus and method for centrally locating the dilation balloon catheter assembly within a stenosed region to ensure proper engagement between the stenosis and the dilation balloon. The catheter assembly can be combined with a stent to form a stenosis treatment device. More specifically, the stent can be disposed about the balloon to engage the stent with a stenosis. It is desirable to have an apparatus and method for medial inflation of a dilation balloon to evenly expand the stent. Preferably, proper medial inflation and location of the dilation balloon in the stenosed region forms a “dog bone” shape. The “dog bone” shape results as the stenosis compresses evenly on the central portion of the dilated balloon and/or stent. This balloon inflation dynamic can limit stent migration along the balloon and thereby minimize any misplacement in stent deployment. Accordingly, it is desirable to provide for consistent medial inflation of the dilation balloon such that the balloon expands evenly and radially from a central point, thus avoiding uneven distortions in the dilation balloon as it is inflated.

In another preferred embodiment, the first marker and the second marker are disposed within the holding volume. In addition, at least one of the first marker and the second marker are radiopaque and/or radiographic. Moreover, the exterior surface of the wall of the catheter defines a first diameter outside the holding volume and a second diameter inside the holding volume. Preferably, the second diameter is smaller than the first diameter and the catheter includes a taper portion between the first and second diameter.

Another preferred embodiment according to the present invention provides a fluid delivery device. The fluid delivery device can include an elongated member having a proximal end and a distal end defining a first lumen and a second lumen spaced apart along a longitudinal axis. The first lumen is preferably configured to convey a fluid, and the member preferably has an opening disposed between the proximal and distal ends in fluid communication with the lumen. The delivery device further preferably includes a first radiopaque and/or radiographic marker and a second radiopaque and/or radiographic marker. The first marker and the second marker are preferably disposed along the longitudinal axis and spaced from one another so as to be substantially equidistant from the opening.

Another preferred embodiment according to the present invention provides a method of engaging a stenosis with an inflatable member having a first end and a second end in which the inflatable member has disposed therein at least a portion of a tubular member having a first radiopaque and/or radiographic marker and a second radiopaque and/or radiographic marker spaced along a longitudinal axis of the tubular member. The method preferably includes locating the first and second markers equidistantly about a portion of the stenosis such that the inflatable member is substantially centered along the length of the portion of the stenosis. The method further preferably includes: flowing a fluid in a channel of the tubular member along the longitudinal axis and introducing a sufficient amount of the fluid into the inflatable member through an opening of the tubular member to expand the inflatable member substantially radially and engage the stenosis. Another embodiment further includes disposing a stent about the inflatable member such that introducing a sufficient amount of fluid into the inflatable member further engages the stent with the stenosis.

Another preferred embodiment provides a method of dilating a stenosis in which the method can be achieved by locating a first marker of a catheter assembly to one side of a portion of a stenosis and locating a second marker on the opposite side of the portion such that the first and second markers are generally equidistant from the portion of the stenosis. The method further includes disposing a fluid fill opening of an inflatable member generally equidistant between the first and second markers, and expanding the inflatable member via the fluid fill opening substantially equally longitudinally and radially about the central region to engage and apply an expansion force to the portion.

### BRIEF DESCRIPTION OF THE DRAWINGS

The accompanying drawings, which are incorporated herein and constitute part of this specification, illustrate a preferred embodiment of the invention, and, together with the general description given above and the detailed description given below, serve to explain the features of the invention.

FIG. 1 is an illustrative perspective view of an embodiment of a balloon catheter assembly.

FIG. 1A is an isometric view of the proximal end of the assembly of FIG. 1.

FIG. 1B is a geometric plan view of the assembly of FIG. 1.

FIG. 2 is a detailed portion of the distal end of the assembly of FIG. 1.

FIG. 2A is a detailed portion of the assembly of FIG. 2.

FIG. 3 is a cross-sectional detail of the assembly of FIG. 2.

FIG. 3A is perspective view of a portion of the assembly of FIG. 2.

FIG. 4 is an illustrative example of the assembly of FIG. 1 used in a stenosis treatment procedure.

### MODE(S) FOR CARRYING OUT THE INVENTION

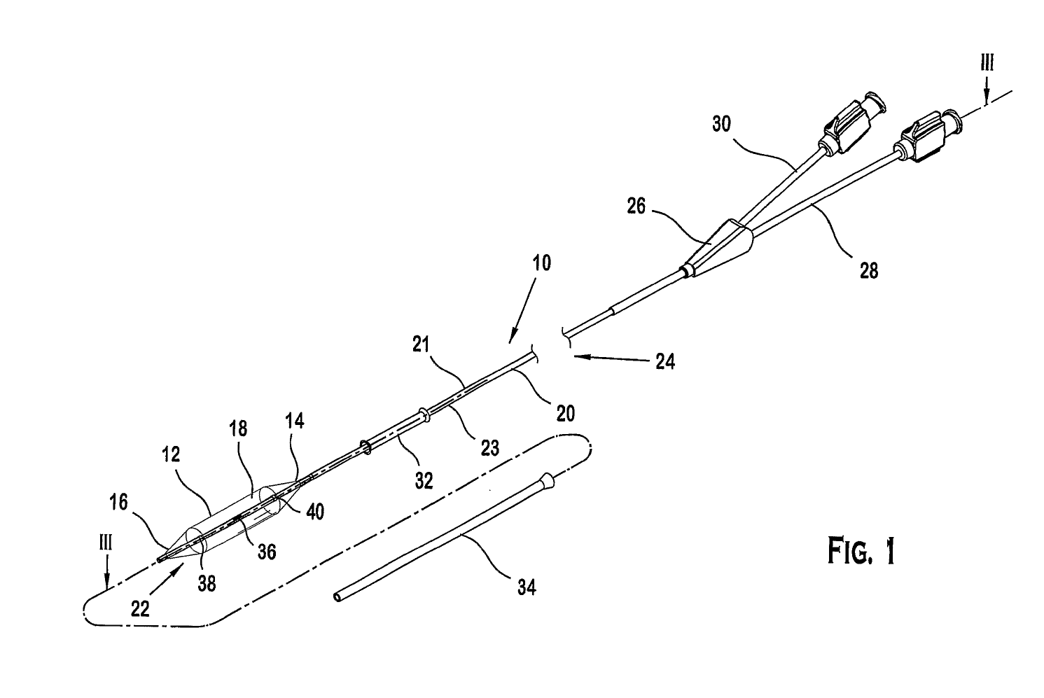

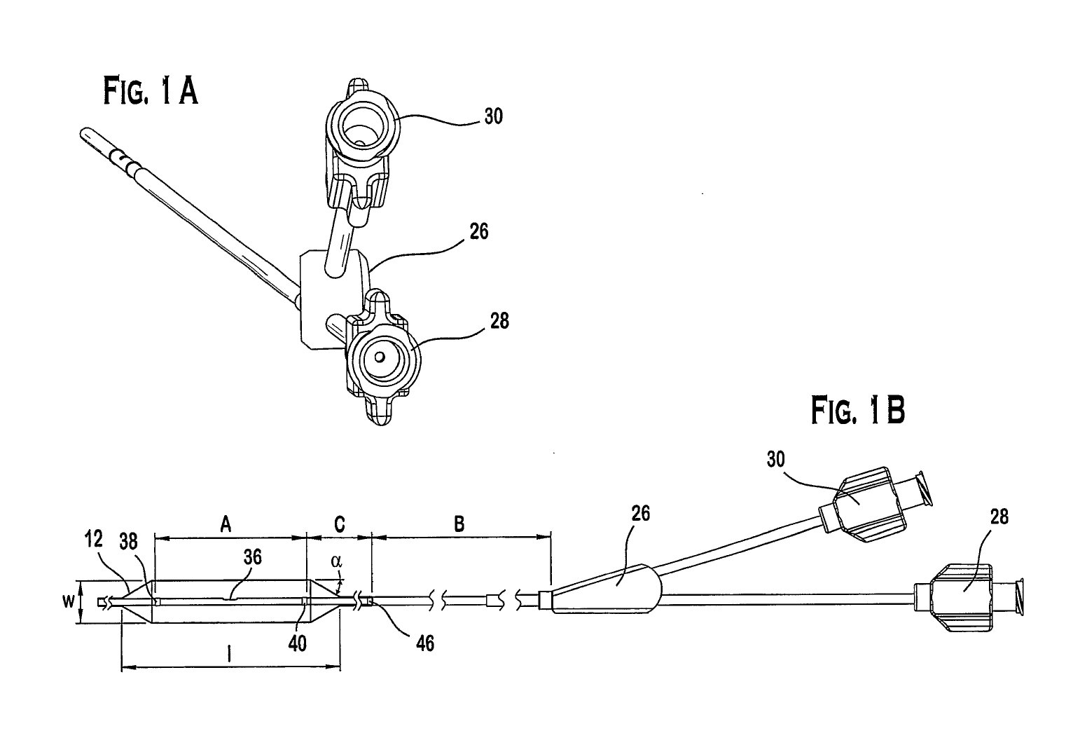

FIG. 1 shows a preferred embodiment of a catheter assembly 10 for engaging a stenosis. More specifically, the catheter assembly 10 can be configured for angioplasty procedures in which an inflatable member or balloon 12 is introduced into a blood vessel for engagement with a diseased portion of the blood vessel such as, for example, a stenosis or for engagement with an implantable prosthesis such as, for example, a stent or stent-graft. The catheter assembly 10 can be further configured for introducing an implant or stent (not shown) into the blood vessel to treat the stenosis. The stent can be disposed about the balloon 12 and the catheter assembly 10 can deliver and position the stent in engagement with the stenosis for implantation. Alternatively, the stent can be delivered to the stenosis independently of the catheter assembly 10. The catheter assembly 10 can subsequently engage the stent at the stenosis site and inflate the balloon 12 to expand the stent for engagement with the stenosis.

Generally, the catheter assembly 10 includes a catheter 20 having a proximal portion 24 a distal portion 22. The catheter 20 preferably is an elongated tubular member having a wall 21 forming a exterior surface 23 and an interior 25 surface (not shown) defining a longitudinal axis III-III. The catheter 20 is preferably formed by extrusion of a thermoplastic material such as, for example, PEBAX 7300® thermoplastic material with a gel content of 9 percent or less compounded with 10 percent Bismuth Subcarbonate. Preferably disposed at the proximal portion 24 is a connector 26 having a first port 28 for introducing a guide wire into the catheter 20 and a second port 30 for introducing a fluid. Disposed at the distal portion 22 of the catheter 20 is the dilation balloon 12. The dilation balloon 12 is preferably disposed about the distal portion 22 of the catheter 20 so as to locate an opening 36 in the catheter 20 within the holding volume 18 of the balloon 12. Fluid is exchanged between the balloon 12 and the catheter 20 through the opening 36 to inflate and deflate the balloon 12. To assist an operator in locating the balloon 12 along a stenosis or other targeted region, the catheter 20 can include first and second, preferably radiographic and/or radiopaque, markers 38, 40 along the distal portion 22 inside the holding volume 18 of the balloon 12.

The balloon 12 of catheter assembly 10 preferably has a first end 14, a second end 16 to define the holding volume 18 therebetween. The first and second ends 14, 16 can be disposed about the catheter 20. Preferably, the first end 14 and second end 16 of the balloon 12 are sealed about the catheter 20 so as to enclose a distal portion 22 of the catheter 20 within the holding volume 18 in a fluid tight manner. For example, the first and second ends 14, 16 can be thermally bonded to the exterior surface 23 of the catheter 20 to form a fluid tight seal. Alternative bonding techniques can be used to seal the ends 14 and 16 to the catheter 20 such as, for example, laser or adhesive bonding techniques. In addition, the balloon 12 can be coupled to the catheter 20 in any other manner to enclose the distal portion 22 of the catheter 20 within the holding volume 18 in a fluid tight manner. The balloon 12 is preferably constructed from a nylon material, such as, Nylon 12 or Nylon 11, or alternatively from other suitable thermoplastic polymers such as, for example, polyether block amide (PEBA), polyethylene, polyethylene terephthalate (PET). Moreover, the balloon can be a composite material balloon formed from a combination of Nylon and other polymers or a combination of ultra high molecular weight polyethylene by itself or with PET. Preferably, the balloon 12 defines a sufficient strength in an inflated state so as to dilate or expand a stent or blood vessel.

One technique for forming the balloon 12 includes blow molding a Nylon or PET tube under heat in a mold to form the desired shape, for example, a circular cylindrical body with two conical tapered ends. The formed balloon 12 can be disposed over and thermally bonded to the catheter 20. U.S. Pat. No. 5,755,690 describes one method for forming a multiple layer high strength balloon for dilation catheter in which a parison, of orientable semicrystalline polymer such as, for example PET, is disposed within a mold with one end of the parison sealed and the other end secured to a fluid source such as, for example, a gas. The parison is axially drawn and radially expanded within the mold to form an expanded balloon. The expanded balloon can then be exposed to a heat step in order to increase crystallinity in the balloon for dimensional stability. The balloon can then be removed from the mold and disposed about the catheter and thermally bonded thereto. Alternatively to thermally bonding the balloon 12, an adhesive can be employed to bond the balloon 12 to the catheter 20.

The distal portion 22 and the proximal portion 24 of the catheter 20 are preferably formed as a unitary construction joined together by a transition section 46. Alternatively, the distal portion 22 and the proximal portion 24 can be distinct elements mechanically joined together by the transition 46. Preferably, the outer diameter of the proximal portion 24 is larger than the outer diameter of the distal portion 22 of the catheter 20. The transition section 46 is preferably tapered from the proximal portion 24 to the distal portion 22. Alternatively, transition section 46 can have a constant diameter to join the proximal portion 24 to the distal portion 22 thereby forming a step transition from the proximal portion 24 to the distal portion 22.

The connector 26 disposed at the proximal end 24 of the catheter 20 can be coupled to the catheter 20 by any suitable techniques such as, for example, interference fit, thread connection or press fit. The connector 26 is preferably disposed proximal of the balloon 12. The connector 26 is configured for introducing a fluid, guide wire or any other instrumentation into the catheter 20. Specifically, the connector 26 includes a first port 28 configured for receipt of a guide wire (not shown) to be inserted along the vein or artery of the patient. The catheter assembly 10 can be disposed about the guide wire so that an operator can guide the assembly 10 along the wire to locate the assembly to a desired location relative to the stenosis within the vein or artery. More specifically and preferably, the balloon 12 can be generally centered across the stenosed lesion. The first port 28 is preferably aligned parallel to or coaxial with the longitudinal axis III-III of the catheter 20.

The connector 26 can further include a second port 30 configured to connect to a fluid source (not shown). The fluid source can be, for example, a syringe or other pump/vacuum device for delivery of a fluid. The fluid is preferably a liquid and can be, for example, a dye, a saline solution or any other contrast fluid to inflate the balloon 12. Shown in FIG. 1A is another embodiment of the connector 26. The second port 30 can be configured for receipt of a syringe as a fluid source to inject and withdraw fluid through the assembly 10. The second port 30 of FIG. 1 is preferably in fluid communication with the first port 28 within the connector 26, however the connector 26 can be configured so as to isolate the fluids from the second port 30 with the first port 28. The port 30 can form an angle incident with the catheter 20. Preferably, the port 30 forms an acute angle incident to the longitudinal axis III-III of the catheter 20 in the direction of fluid flow moving distally away from an operator. During a procedure, the fluid is preferably introduced into the second port 30 and further into the catheter 20. The fluid is discharged from an opening 36 in the distal portion 22 of the catheter 20 and into the holding volume 18 to expand the balloon 12. Preferably, the fluid is introduced into the balloon 12 to expand the balloon radially from the opening 36, along and about the longitudinal axis III-III. The port 30 can also be used to extract fluid from and deflate the balloon 12. Fluid can be drawn from the balloon 12 into the catheter 20 preferably through the opening 36 and returned to the fluid source via the connector 26 and port 30.

FIG. 1 and FIG. 1B show the balloon 12 in an inflated state with FIG. 1B providing particular geometric relationships of the assembly 10. In the inflated state, the balloon 12 is shown as a substantially tubular or cylindrical member along the longitudinal axis III-III. In a plane perpendicular to the longitudinal axis III-III, the balloon 12 defines a cross-sectional section that is preferably circular, however other cross-sections are possible such as, for example, oval, multi-lobed or other polygons. The width w (preferably the diameter) of the balloon 12, as seen in FIG. 1B, can range from about 1 millimeter to about 40 millimeters, preferably range from about 1 millimeter to about 26 millimeters and even more preferably range from about 3 millimeters to about 20 millimeters, and the length l of the balloon 12 can range from about 10 millimeters to about 120 millimeters. Each end of the balloon 12 is preferably conical so as to preferably defines a cone angle α relative to a line parallel to the longitudinal axis III-III. The cone angle can range from about five degrees (5°) to about thirty degrees (30°) depending upon the length l of the balloon. The dimensions A, B and C of the catheter 20 can vary along with the width w and length l of the balloon 12. More specifically, dimension A measured from the first preferably radiopaque and/or radiographic marker 38 to the second preferably radiopaque and/or radiographic marker 40 can be of any suitable length and preferably any one of about, 10 millimeters, 15 millimeters, 20 millimeters, 30 millimeters, 40 millimeters, 60 millimeters, 80 millimeters, 100 millimeters, to about 120 millimeters in length. Dimension B, measured from the transition section 46 to the connector 26 can preferably be of any suitable length and preferably, any one of about, 40 centimeters, 75 centimeters, 115 centimeters, 130 centimeters, to about 140 centimeters in length. Dimension C measured from the transition section 46 to the second marker 40 can preferably be any one of about, 10 millimeters, 15 millimeters, to about 20 millimeters in length.

Referring again to FIG. 1, the catheter assembly 10 can also include a deflator 32 that is preferably a sliding member 32 disposed about the outer surface 23 of the catheter 20. The sliding member 32 can be permitted to slide along the catheter 20 between the distal and proximal portions 22, 24. The sliding member 32 can be configured to assist in deflating the balloon member 18 by passing over the balloon 12 to displace any fluid and/or air in the holding volume 18. The sliding member 32 can include a central channel through which the balloon 12 and the catheter 20 can pass. The body of the sliding member 32 is preferably substantially spool shaped to provide a low profile and easy handling for the operator; however, other geometries are possible permitting manual manipulation. The catheter assembly 10 can also include a removable cap 34. The cap 34 can engage and disengage from the balloon 12 and the distal portion 22 of catheter 22 to protect the balloon 12 from damage when not in use.

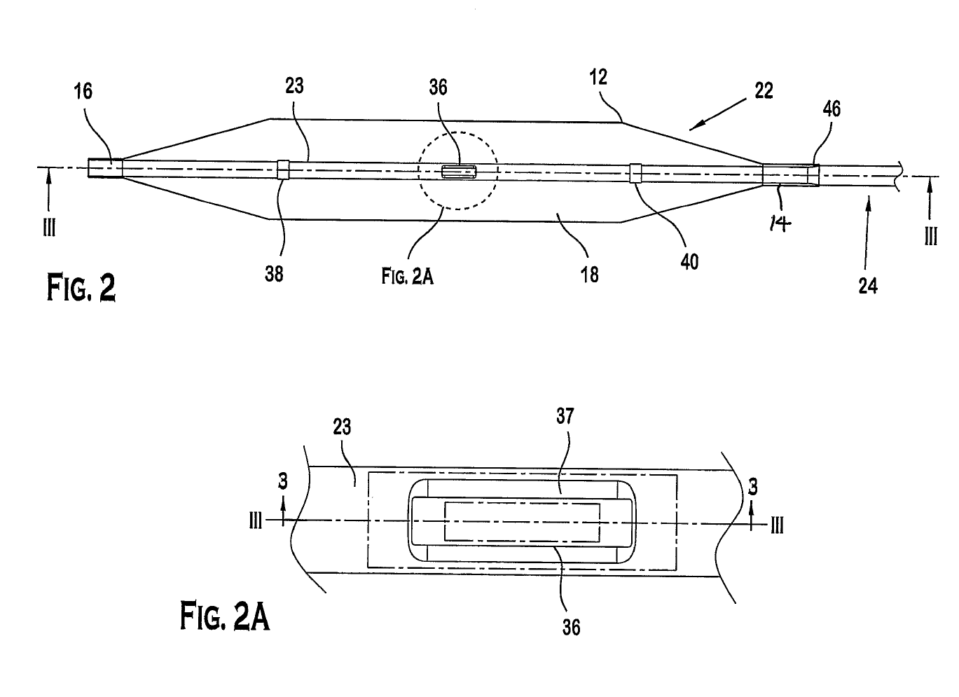

FIG. 2 shows an enlarged view of the distal portion 22 of the catheter 20 sealed within the balloon 12. The distal portion 22 of the catheter 20 further includes the opening 36. Preferably, first and second ends 14, 16 of the balloon 12 are secured about the catheter 20 so as to be equidistantly spaced from the opening 36 and thus place the opening 36 in a substantially central location within the holding volume 18 of the balloon 12. Any fluid introduced into the catheter 20 can be discharged through the opening 36 to inflate the balloon 12 from an initial deflated state or volume (not shown) to a substantially inflated state or volume (as shown in FIG. 2).

Shown in FIG. 2A is the plan view detail of the opening 36. The opening 36 is preferably rectangular and elongated in the direction of the longitudinal axis III-III so as to deliver and evacuate a sufficient volume of fluid to respectively inflate and deflate the balloon 12. The opening 36 can further include a chamfer or transition 37 from the interior of the catheter 20 to the outer surface 23, and the edges of the opening 36 along the outer surface 23 are preferably rounded to assist in achieving the desired flow characteristics. Where, for example, the opening 36 is rectangular, the dimensions of opening 36 can measure about 0.2 centimeters in length and about 0.02 centimeters in width. Generally, opening 36 can have any dimensioned geometry and transition characteristics such as, for example, a substantially circular, oval or polygonal, so long as the desired flow characteristics are obtained for the rapid inflation and deflation of the balloon 12. Preferably the opening 36 is dimensioned and configured in a manner that provides for the inflation and deflation of the balloon 12 within a time period that minimizes the time for which the blood vessel may be occluded by the balloon 12. As described above, the dimensions of the catheter 20 can vary with the dimensions of the balloon 12. Accordingly, the dimensions of the opening 36 and the balloon 12 can be such as to define a relationship over various configurations of the catheter 20. Specifically, in one preferred embodiment, the area of the opening 36 and the fully expanded holding volume 18 of the balloon 12 can define a ratio of area to volume. This ratio can be constant over the various configurations of the catheter 20. Alternatively, the ratio of the area of the opening 36 and the fully expanded holding volume 18 of the balloon 12 can be variable over the various configurations of the catheter 20.

The centralized location of the opening 36 shown in FIG. 2 relative to the balloon 12 can provide a fluid distribution within the balloon 12 to facilitate the even and radial expansion of the balloon 12 from the deflated state to the inflated state. More specifically, the fluid discharging from the substantially central point within the holding volume 18 of the balloon 12 engages interior surfaces of the balloon equally radially and evenly along the direction of the longitudinal axis III-III. Thus, uneven concentrations of fluid or waves which can distort the shape of the balloon 12 are minimized or otherwise avoided. This can ensure that a target area (e.g., stenosis or stent) is engaged fully and evenly by the balloon 12 or stent to produce the preferable “dog bone” shape the balloon 12. In a case where the balloon 12 is being used to implant a stent, the centralized expansion of the balloon 12 can ensure that the stent is expanded substantially evenly along its length.

The distal portion 22 of the catheter 20 further includes the first marker 38 and the second marker 40 disposed on the exterior surface 23 of the catheter 20. Preferably, the markers 38, 40 are made of a radiopaque and/or radiographic material such as, for example, 18 Karat Gold, platinum, tantalum, BaSO₄ Iridium to make the catheter 20 or at least the distal portion 22 visible under fluoroscopic observation. The markers 38, 40 can be used by an operator to guide the catheter assembly 10 under fluoroscopic observation to a desired location within the blood vessel. The first and second radiopaque and/or radiographic markers 38, 40 are preferably spaced apart and located along the longitudinal axis III-III such that the markers are equidistantly spaced from the opening 36. More preferably, the markers 38, 40 are disposed within the holding volume 18. Because the first and second ends 14, 16 of the balloon 12 are also preferably centered about the opening 36, the first and second markers 38, 40 can facilitate the centering of the balloon 12 with respect to the target area. In particular, a clinician can utilize the radiopaque markers 38, 40 under fluoroscopic observation to center the opening 36 along the length of the target area, such as a stenosed lesion, and because of the fixed relation of the balloon ends 14, 16 to the opening 36, the balloon is thereby preferably centered with respect to the target region for properly engaging the length of the target region.

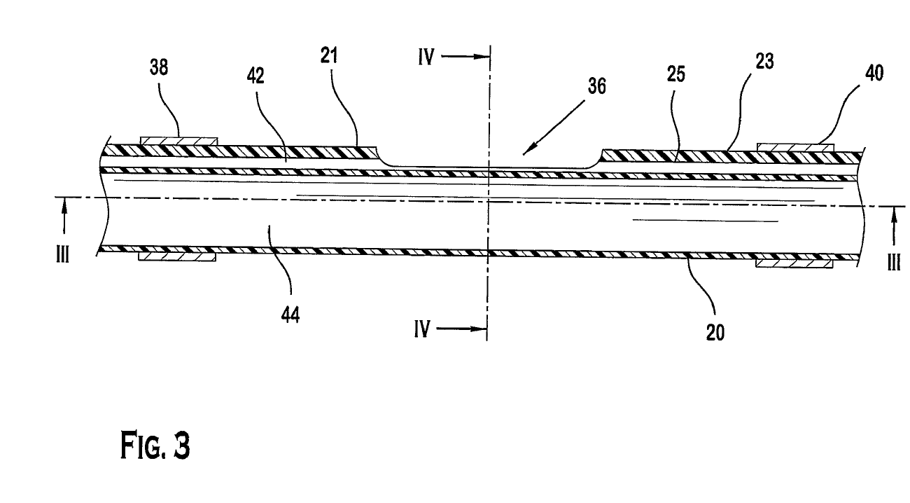

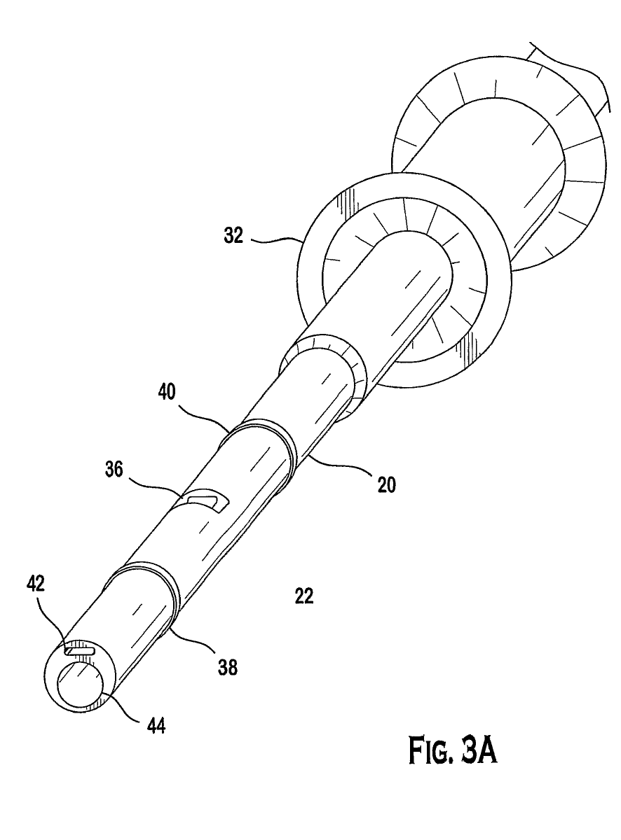

Shown in FIG. 3 is a cross-sectional view of a portion of the distal portion 22 of the catheter 20. The interior surface 25 of the wall 21 forming the catheter 20 can further define a first channel or lumen 42, preferably parallel to the longitudinal axis III-III. The lumen 42 can extend from the distal portion 22 into the proximal portion 24 of catheter 20 for communication with the second port 30 of the connector 26 in order to exchange a fluid, preferably a liquid, between the balloon 12 and the fluid source for inflation/deflation of the balloon 12. The inner diameter of the lumen 42 is dimensioned to provide a sufficient flow of fluid given the delivery pressures from the fluid source such as, for example, a syringe. The inner diameter of the first lumen 42 can remain constant over the entire length of the catheter 20 or alternatively, the inner diameter of the first lumen 22 can change over the length of the catheter 20. The lumen 42 is preferably offset from the centerline longitudinal axis III-III of the catheter 20.

To facilitate fluid exchange between the balloon 12 and the catheter 20, the lumen 42 is in fluid communication with the holding volume 18 via the opening 36 shown in FIGS. 2 and 3. More specifically, the opening 36 is positioned relative to the lumen 42 so as to define a fluid path having an angle incident to the longitudinal axis III-III. Fluid conveyed along the lumen 42 can be discharged from the opening 36 and into the holding volume 18 to expand the balloon 12. Preferably, the flow path is substantially orthogonal to the longitudinal axis III-III to radially disperse the fluid from a substantially central portion of the holding volume 18. Alternatively, the opening 36 can be positioned and configured so as to define a fluid path having an acute angle with longitudinal axis III-III so long as the fluid path can be dispersed from a substantially central portion of the holding volume 18.

Shown in FIG. 3A is an end view of the catheter 20. Preferably, the cross-section of the first lumen 42 is substantially rectangular and more preferably is crescent shape to convey an adequate flow of fluid to and from the holding volume 18. The first lumen 42 can be dimensioned and configured so as to adequately fit within the overall size constraints of the catheter 20 such as, for example, the outer diameter of the catheter 20 and the demands on cross-sectional area of the catheter 20 to accommodate any additional lumen. The cross-sectional area of the lumen 42 can define other geometries such as substantially circular, for example, so long as the lumen 42 is dimensioned to convey the adequate fluid flow. In a preferred embodiment, the lumen 42 is sealed at the distal end so as to provide a sufficient discharge pressure at the opening 36 to promote the even radial expansion of the balloon 12. Generally, the balloon 12 is rated for an operational pressure ranging from about 4 atmosphere (atm.) to about 8 atmosphere (atm.) and is more preferably about 8 atm., which corresponds to an operational delivery pressure of about 125 psi. Depending on the size of the balloon 12, the balloon 12 can further be configured for rated burst pressures ranging from about 8 atm. to about 16 atm. Alternatively, the lumen 42 can have multiple discharge openings so long as a sufficient discharge pressure is provided at the opening 36.

Fluid in the holding volume 18 can be drawn through the opening 36 and into the lumen 42 to deflate the balloon 12. In addition to facilitating the radial expansion of the balloon 12, the central positioning of the opening 36 relative to the holding volume 18 can maximize the time for which the opening 36 remains patent as fluid is drawn through the opening 36 and the balloon 12 collapses about the distal end 22 of the catheter 20 and eventually over opening 36. Accordingly, the positioning of the opening 36 can control the efficiency of deflation of the balloon 12. The efficiency of balloon deflation can define the time required to deflate the balloon 12 thereby defining the period that an inflated balloon 12 blocks or restricts the flow of blood through the blood vessel. Generally, it is desired that the time period for which the expansion of balloon 12 blocks blood flow through the blood vessel be minimized.

The catheter 20 shown in FIG. 3 preferably includes a second channel or lumen 44 distinctly defined by the wall 21 extending parallel to the longitudinal axis III-III and the first lumen 42. The second lumen 44 is dimensioned and configured to receive a guide wire upon which the catheter assembly 10 can translate. The second lumen 44 separates the guide wire from the fluid flow in the lumen 42, thereby eliminating interference with the flow or pressure characteristics of the fluid by the presence of the guide wire. Preferably, the second lumen 44 extends from the distal end to the proximal end of the catheter 20 for communication with the first port 28 of the connector 26. The second lumen 44 is preferably dimensioned and configured to receive the guide wire from the port 28. The guide wire can be a conventional surgical guide wire such as, for example, stainless steel type 302 or 304 having an outer diameter of about 0.25 millimeter. The first and second lumen 42, 44 can alternatively be defined by distinct tube members within a single larger catheter tube (not shown).

The inner diameter of the second lumen 44 can remain constant over the entire length of the catheter 20 or alternatively, the inner diameter of the second lumen 44 can change over the length of the catheter 20 to accommodate space demands on the overall cross-sectional area of the catheter 20. Preferably, the overall cross-sectional area of the catheter 20 remains constant over the various configurations of the catheter 20 discussed above. Alternatively, the overall cross-sectional area of the catheter 20 can vary proportionally with any one or more of the dimensions defining the catheter 20 such as, for example, the catheter's overall length or the lengths A, B or C described above. Shown in FIG. 3A is the cross-section of the lumen 44 as being substantially circular to provide the guide wire a substantially smooth wall through which to pass. Alternatively, other geometries are possible such as rectangular, oval or any other configuration so long as the lumen 44 is dimensioned to permit passage of the guide wire.

The second lumen 44 is preferably offset from the centerline longitudinal axis III-III of the catheter 20 to accommodate the dimension and configuration of the first lumen 42 for the delivery of the proper operating pressure for inflating the balloon 12. The catheter can be dimensioned to accommodate additional lumen to provide channels for the insertion of other fluids or devices such as, for example, a third lumen to carry a temperature probe (not shown).

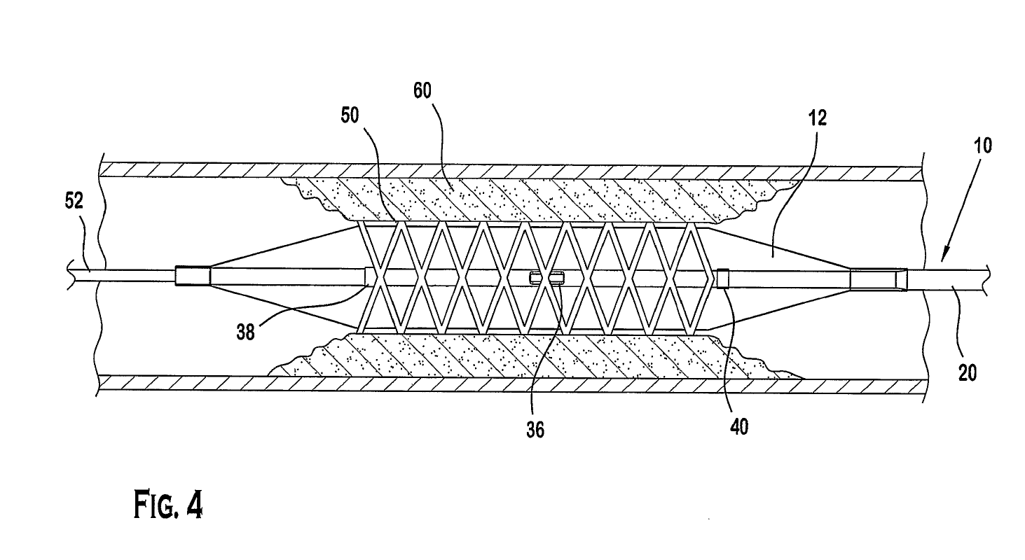

Shown in FIG. 4 is an illustrative depiction of a stent delivery procedure in which the preferred embodiment of the catheter assembly 10 described above is locating and positioning a stent 50 along a stenosis 60 for expansion of the stenosed lesion and blood vessel 62. The catheter assembly 10 is preferably disposed about a guide wire 52, and an operator using the assembly 10 under fluoroscopy observation can align the balloon 10 and the stent 50 with the stenosis and further identify a portion of the stenosis 60 to which a direct expansion force using the balloon 12 of the assembly 10 can be applied. Preferably, the identified portion is the central portion of the stenosis 60. Accordingly, the operator slides the catheter assembly 10 along the guide wire 52 to align the radiopaque markers 38, 40 equidistantly about the central portion of the stenosis 60 and thereby align a substantially central region of the balloon 12 with the central portion of the stenosis.

A contrast fluid can be channeled along the catheter 20 and introduced into the holding volume 18 of the balloon 12 through the opening 36 to fully dilate the balloon 12 and the stent 50 as shown. The preferably fixed centralized relation of the opening 36 to the markers 38, 40 aligns the opening 36 with the identified portion of the stenosis to be expanded, and with the opening 36 being preferably centrally located in the holding volume 18, the balloon 12 and stent 50 are preferably evenly and radially expanded about the central region of the balloon 12 into engagement with the stenosis to apply expansion forces at least to the identified portion.

The various configurations of the catheter assembly 10 described herein provide numerous advantages in the performing angioplasty and stent delivery procedures. The catheter 20 preferably includes two spaced apart lumen for separately carrying a guide wire and an inflation fluid. The separately dedicated lumen can facilitate delivery of the inflation fluid at the proper operating pressure to expand the inflation balloon 12 by minimizing or eliminating interference of the guide wire with the fluid flow or delivery pressure. The opening 36 of catheter 20 is preferably disposed centrally within the holding volume 18 to facilitate central and localized fluid delivery within the holding volume 18 to promote even radial expansion of the balloon 12. The even radial expansion of the balloon 12 can ensure proper engagement between the balloon 12 and the stent or stent graft so as to evenly radially expand the stent device and prevent migration of the stent device along the balloon 12. In addition, the centralized location of the opening 36 relative to the holding volume 18 can increase the efficiency of the balloon deflation by maximizing the patency of the opening 36 to withdraw fluid from the balloon 12 while minimizing the time balloon remains in an expanded state to occlude the blood vessel being treated. In addition, the markers 38, 40 are preferably located within the holding volume 18 and relative to the opening 36 of the catheter 20 to provide the necessary visual indicators to center the balloon 12 relative to the target area or region. The radiopaque and/or radiographic markers 38, 40 assist in properly locating the balloon and/or stent or stent graft relative to the center of a target region or center.

While the present invention has been disclosed with reference to certain embodiments, numerous modifications, alterations and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof. As used herein, the singular form of “a,” “an,” and “the” include the plural referents unless specifically defined as only one.

What is claimed is:

1. A catheter assembly for engaging a stenosis, the assembly comprising:
a catheter including a wall having a proximal end and a distal end disposed along a longitudinal axis, the wall having an interior surface and an exterior surface, the interior surface defining a first lumen and a second lumen spaced apart and disposed about the longitudinal axis, the wall defining an opening extending between the inner surface and the exterior surface, the opening being in communication with the first lumen so as to define a flow path having an angle incident to the longitudinal axis;
a first marker disposed about the exterior surface along the longitudinal axis; and
a second marker disposed about the exterior surface along the longitudinal axis, the first and second markers being spaced from one another along the longitudinal axis so as to be substantially equidistant from the opening; and
a balloon having a first end and a second end defining a holding volume therebetween, the first end and the second ends being sealed about the exterior surface, the opening is disposed within the holding volume so as to place the first lumen in sealed fluid communication with the holding volume, the first and second ends being spaced substantially equidistantly about the opening along the longitudinal axis.

2. The catheter assembly of claim 1, wherein the holding volume defines a first volume and a second volume larger than the first volume, the opening being configured so as to alter the holding volume between the first and the second volumes substantially radially about the opening.

3. The catheter assembly of claim 2, wherein the balloon has an operational pressure of about 8 atm. so as to define a fluid delivery pressure at the opening to alter the holding volume between the first and the second volumes.

4. The catheter assembly of claim 1, wherein the balloon has a width ranging from about 1 millimeter to about 40 millimeters.

5. The catheter-assembly of claim 1, wherein the balloon has a width ranging from about 1 millimeter to about 26 millimeters.

6. The catheter-assembly of claim 1, wherein the balloon has a width ranging from about 3 millimeter to about 20 millimeters.

7. The catheter-assembly of claim 1, wherein the balloon has a length ranging from about 10 millimeters to about 120 millimeters.

8. The catheter-assembly of claim 1, further comprising a connector disposed about the exterior surface proximate the balloon, the connector having a first port and a second port in communication with the second lumen, the first port being in fluid communication with the first and the second lumen.

9. The catheter assembly of claim 8, wherein the first port defines an angle incident to the first lumen and the second port is substantially coaxial with the second lumen.

10. The catheter-assembly of claim 1, wherein the first marker and the second marker are disposed within the holding volume.

11. The catheter-assembly of claim 1, wherein at least one of the first marker and the second marker comprises at least one of a radiopaque and radiographic material.

12. The catheter-assembly of claim 1, wherein the exterior surface of the wall defines a first diameter outside the holding volume, the exterior surface defining a second diameter inside the holding volume, the second diameter being smaller than the first diameter, the exterior surface including a taper portion between the first and second diameter.

13. The catheter-assembly of claim 1, further comprising a deflator disposed about the exterior surface, the deflator having a first position proximate the balloon and a second position distal the balloon, the deflator being configured to translate along the longitudinal axis from the first position to the second position so as to alter the holding volume.

14. The catheter-assembly of claim 1, further comprising a cap engaged with the catheter such that the balloon is disposed inside the cap.

15. The catheter-assembly of claim 1, further comprising a stent disposed about the balloon, the balloon being configured to engage the stent with a stenosis.

16. A fluid delivery device comprising:
an elongated member having a proximal end and a distal end defining a first lumen and a second lumen spaced apart from the first lumen along a longitudinal axis, the first lumen being configured to convey a fluid, the member having an opening disposed between the proximal and distal ends, the opening being in fluid communication with the first lumen; and
a first marker and a second marker, the first marker and second marker being disposed about the member and spaced from one another so as to be substantially equidistant from the opening.

17. The fluid delivery device of claim 16, wherein the first lumen defines a substantially crescent shaped cross-sectional area perpendicular to the longitudinal axis.

18. The fluid delivery device of claim 16, wherein the first lumen defines a substantially rectangular cross-sectional area perpendicular to the longitudinal axis.

19. The fluid delivery device of claim 16, wherein the opening comprises a substantially rectangular opening.

20. The fluid delivery device of claim 16, wherein the first marker and the second marker are a radiopaque and radiographic material.

21. The fluid delivery device of claim 16, wherein the second lumen is disposed about the guidewire.

22. The fluid delivery device of claim 16, further comprising a connector disposed about the exterior, the connector having a first port in communication with the first lumen and a second port in communication with the second lumen.

23. The fluid delivery device of claim 16, wherein the elongated member includes a proximal portion and a distal portion, the proximal portion having a first diameter and the distal portion having a second diameter, the second diameter being smaller than the first.

24. The fluid delivery device of claim 23 wherein the proximal portion tapers down to the distal portion.

25. A method of expanding a stenosis, the method comprising:
locating a first marker of a catheter assembly to one side of a portion of a stenosis and locating a second marker on the opposite side of the portion such that the first and second markers are equidistant from the portion of the stenosis so as to align a substantially central region of an inflatable member with the portion; and
introducing a sufficient amount of the fluid into the inflatable member to expand the inflatable member substantially equally and radially about the central region to engage and apply an expansion force to the portion.

26. The method of claim 25, wherein the introducing comprises interposing a stent between the inflatable member and the portion.

27. A method of dilating a stenosis, the method comprising:
locating a first marker of a catheter assembly to one side of a portion of a stenosis and locating a second marker on the opposite side of the portion such that the first and second markers are generally equidistant from the portion of the stenosis;
disposing a fluid fill opening of an inflatable member generally equidistant between the first and second markers; and
expanding the inflatable member via the fluid fill opening substantially equally longitudinally and radially about the central region to engage and apply an expansion force to the portion.
